SEQUENCE LISTING (14 sequences)

SEQ ID NO: 1 - protein, 357 aa, Homo sapiens
MENKAMYLHTVSDCDTSSICEDSFDGRSLSKLNLCEDVSRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASKDILLGPWDMVLAQG

SEQ ID NO: 2 - protein, 270 aa, Homo sapiens
MENKAMYLHTVSDCDTSSICEDSFDGRSLSKLNLCEDGPCHKRRASICCTQLGSLSALKHAVLGLYLLVFLILVGIFILAGPPGPKGDQGDEGKEGRPGIPGLPGLRGLPGERGTPGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 3 - protein, 495 aa, Homo sapiens
MENKAMYLHTVSDCDTSSICEDSFDGRSLSKLNLCEDGPCHKRRASICCTQLGSLSALKHAVLGLYLLVFLILVGIFILAVSRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 4 - protein, 400 aa, Homo sapiens
MENKAMYLHTVSDCDTSSICEDSFDGRSLSKLNLCEDGPCHKRRASICCTQLGSLSALKHAVLGLYLLVFLILVGIFILAVSRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASKDILLGPWDMVLAQG

SEQ ID NO: 5 - protein, 217 aa, Homo sapiens [sequence omitted]

SEQ ID NO: 6 - protein, 189 aa, Homo sapiens
TRNVNRLNESFRDLQLRLLQAPLQADLTEQVWKVQDALQNQSDSLLALAGAVQRLEGALWGLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASGVEA

SEQ ID NO: 7 - protein, 357 aa, Homo sapiens; identical to SEQ ID NO: 1

SEQ ID NO: 8 - DNA, 1514 bp, Homo sapiens
tttattttatacggactggcggcgagagcagctgcagttcgcatctcaggcagtacctagaggagctgccggtgcctcctcagaacatctcctgatcgctacccaggaccaggcaccaaggacagggagtcccaggcgcacaccccccattctgggtcccccaggcccagacccccactctgccacaggttgcatcttgacctggtcctcctgcagaagtggcccctgtggtcctgctctgagactcgtccctgggcgcccctgcagcccctttctatgactccatctggatttggctggctgtggggacgcggtccgaggggcggcctggctctcagcgtggtggcagccagctctctggccaccatggcaaatgctgagatctgaggggacaaggctctacagcctcagccaggggcactcagctgttgcagggtgtgatggagaacaaagctatgtacctacacaccgtcagcgactgtgacaccagctccatctgtgaggattcctttgatggcaggagcctgtccaagctgaacctgtgtgaggatgtgtccaggccgcgcagctcccctgacgacctgaaggccctgactcgcaatgtgaaccggctgaatgagagcttccgggacttgcagctgcggctgctgcaggctccgctgcaagcggacctgacggagcaggtgtggaaggtgcaggacgcgctgcagaaccagtcagactcgttgctggcgctggcgggcgcagtgcagcggctggagggcgcgctgtgggggctgcaggcgcaggcggtgcagaccgagcaggcggtggccctgctgcgggaccgcacgggccagcagagcgacacggcgcagctggagctctaccagctgcaggtggagagcaacagtagccagctgctgctgaggcgccacgcgggcctgctggacgggctggcgcgcagggtgggcatcctgggcgaggagctggccgacgtgggcggcgtgctgcgcggcctcaaccacagcctgtcctacgacgtggccctccaccgcacgcggctgcaggacctgcgggtgctggtgagcaacgccagcgaggacacgcgccgcctgcgcctggcgcacgtaggcatggagctgcagctgaagcaggagctggccatgctcaacgcggtcaccgaggacctgcgcctcaaggactgggagcactccatcgcactgcggaacatctccctcgcgaaagggccaccgggacccaaaggtgatcagggggatgaaggaaaggaaggcaggcctggcatccctggattgcctggacttcgaggtctgcccggggagagaggtaccccaggattgcccgggcccaagggcgatgatgggaagctgggggccacaggaccaatgggcatgcgtgggttcaaaggtgaccgaggcccaaaaggagagaaaggagagaaaggagacagagctggggatgccagtaaggacattctgctggggccgtgggatatggtgttggcacagggctag

SEQ ID NO: 9 - DNA, 2951 bp, Homo sapiens
tttattttatacggactggcggcgagagcagctgcagttcgcatctcaggcagtacctagaggagctgccggtgcctcctcagaacatctcctgatcgctacccaggaccaggcaccaaggacagggagtcccaggcgcacaccccccattctgggtcccccaggcccagacccccactctgccacaggttgcatcttgacctggtcctcctgcagaagtggcccctgtggtcctgctctgagactcgtccctgggcgcccctgcagcccctttctatgactccatctggatttggctggctgtggggacgcggtccgaggggcggcctggctctcagcgtggtggcagccagctctctggccaccatggcaaatgctgagatctgaggggacaaggctctacagcctcagccaggggcactcagctgttgcagggtgtgatggagaacaaagctatgtacctacacaccgtcagcgactgtgacaccagctccatctgtgaggattcctttgatggcaggagcctgtccaagctgaacctgtgtgaggatggtccatgtcacaaacggcgggcaagcatctgctgtacccagctggggtccctgtcggccctgaagcatgctgtcctggggctctacctgctggtcttcctgattcttgtgggcatcttcatcttagcagggccaccgggacccaaaggtgatcagggggatgaaggaaaggaaggcaggcctggcatccctggattgcctggacttcgaggtctgcccggggagagaggtaccccaggattgcccgggcccaagggcgatgatgggaagctgggggccacaggaccaatgggcatgcgtgggttcaaaggtgaccgaggcccaaaaggagagaaaggagagaaaggagacagagctggggatgccagtggcgtggaggccccgatgatgatccgcctggtgaatggctcaggtccgcacgagggccgcgtggaagtgtaccacgaccggcgttggggcaccgtgtgtgacgacggctgggacaagaaggacggagacgtggtgtgccgcatgctcggcttccgcggtgtggaggaggtgtaccgcacagctcgattcgggcaaggcactgggaggatctggatggatgacgttgcctgcaagggcacagaggaaaccatcttccgctgcagcttctccaaatggggggtgacaaactgtggacatgccgaagatgccagcgtgacatgcaacagacactgaaagtgggcagagcccaagttcggggtcctgcacagagcacccttcctgcatccctggggtggggcacagctcggggccaccctgaccatgcctcgaccacaccccgtccagcattctcagtcctcacacctgcatcccaggaccgtgggggccggtcgtcatttccctcttgaacatgtgctccgaagtataactctgggacctactgcccgtctctctcttccaccaggttcctgcatgaggagccctgatcaactggatcaccactttgcccagcctctgaacaccatgcaccaggcctcaatatcccagttccctttggccttttagttacaggtgaatgctgagaatgtgtcagagacaagtgcagcagcagcgatggttggtagtatagatcatttactcttcagacaattcccaaacctccattagtccaagagtttctacatcttcctccccagcaagaggcaacgtcaagtgatgaatttcccccctttactctgcctctgctccccatttgctagtttgaggaagtgacatagaggagaagccagctgtaggggcaagagggaaatgcaagtcacctgcaggaatccagctagatttggagaagggaatgaaactaacattgaatgactaccatggcacgctaaatagtatcttgggtgccaaattcatgtatccacttagctgcattggtccagggcatgtcagtctggatacagccttacctccaggtagcacttaactggtccattcacctagactgcaagtaagaagacaaaatgactgagaccgtgtgcccacctgaacttattgtctttacttggcctgagctaaaagcttgggtgcaggacctgtgtaactagaaagttgcctacttcagaacctccagggcgtgagtgcaaggtcaaacatgactggcttccaggccgaccatcaatgtaggaggagagctgatgtggagggtgacatgggggctgcccatgttaaacctgagtccagtgctctggcattgggcagtcacggttaaagccaagtcatgtgtgtctcagctgtttggaggtgatgattttgcatcttccaagcctcttcaggtgtgaatctgtggtcaggaaaacacaagtcctaatggaacccttaggggggaaggaaatgaagattccctataacctctgggggtggggagtaggaataaggggccttgggcctccataaatctgcaatctgcaccctcctcctagagacagggagatcgtgttctgctttttacatgaggagcagaactgggccatacacatgttcaagaactaggggagctacctggtagcaagtgagtgcagacccacctcaccttgggggaatctcaaactcataggcctcagatacacgatcacctgtcatatcaggtgagcactggcctgcttggggagagacctgggcccctccaggtgtaggaacagcaacactcctggctgacaactaagccaatatggccctaggtcattcttgcttccaatatgcttgccactccttaaatgtcctaatgatgagaaactctctttctgaccaattgctatgtttacataacacgcatgtactcatgcatcccttgccagagcccatatatgtatgcatatataaacatagcactttttactacatagctcagcacattgcaaggtttgcatttaagtt

SEQ ID NO: 10 - DNA, 3980 bp, Homo sapiens
tttattttatacggactggcggcgagagcagctgcagttcgcatctcaggcagtacctagaggagctgccggtgcctcctcagaacatctcctgatcgctacccaggaccaggcaccaaggacagggagtcccaggcgcacaccccccattctgggtcccccaggcccagacccccactctgccacaggttgcatcttgacctggtcctcctgcagaagtggcccctgtggtcctgctctgagactcgtccctgggcgcccctgcagcccctttctatgactccatctggatttggctggctgtggggacgcggtccgaggggcggcctggctctcagcgtggtggcagccagctctctggccaccatggcaaatgctgagatctgaggggacaaggctctacagcctcagccaggggcactcagctgttgcagggtgtgatggagaacaaagctatgtacctacacaccgtcagcgactgtgacaccagctccatctgtgaggattcctttgatggcaggagcctgtccaagctgaacctgtgtgaggatggtccatgtcacaaacggcgggcaagcatctgctgtacccagctggggtccctgtcggccctgaagcatgctgtcctggggctctacctgctggtcttcctgattcttgtgggcatcttcatcttagcagtgtccaggccgcgcagctcccctgacgacctgaaggccctgactcgcaatgtgaaccggctgaatgagagcttccgggacttgcagctgcggctgctgcaggctccgctgcaagcggacctgacggagcaggtgtggaaggtgcaggacgcgctgcagaaccagtcagactcgttgctggcgctggcgggcgcagtgcagcggctggagggcgcgctgtgggggctgcaggcgcaggcggtgcagaccgagcaggcggtggccctgctgcgggaccgcacgggccagcagagcgacacggcgcagctggagctctaccagctgcaggtggagagcaacagtagccagctgctgctgaggcgccacgcgggcctgctggacgggctggcgcgcagggtgggcatcctgggcgaggagctggccgacgtgggcggcgtgctgcgcggcctcaaccacagcctgtcctacgacgtggccctccaccgcacgcggctgcaggacctgcgggtgctggtgagcaacgccagcgaggacacgcgccgcctgcgcctggcgcacgtaggcatggagctgcagctgaagcaggagctggccatgctcaacgcggtcaccgaggacctgcgcctcaaggactgggagcactccatcgcactgcggaacatctccctcgcgaaagggccaccgggacccaaaggtgatcagggggatgaaggaaaggaaggcaggcctggcatccctggattgcctggacttcgaggtctgcccggggagagaggtaccccaggattgcccgggcccaagggcgatgatgggaagctgggggccacaggaccaatgggcatgcgtgggttcaaaggtgaccgaggcccaaaaggagagaaaggagagaaaggagacagagctggggatgccagtggcgtggaggccccgatgatgatccgcctggtgaatggctcaggtccgcacgagggccgcgtggaagtgtaccacgaccggcgttggggcaccgtgtgtgacgacggctgggacaagaaggacggagacgtggtgtgccgcatgctcggcttccgcggtgtggaggaggtgtaccgcacagctcgattcgggcaaggcactgggaggatctggatggatgacgttgcctgcaagggcacagaggaaaccatcttccgctgcagcttctccaaatggggggtgacaaactgtggacatgccgaagatgccagcgtgacatgcaacagacactgaaagtgggcagagcccaagttcggggtcctgcacagagcacccttcctgcatccctggggtggggcacagctcggggccaccctgaccatgcctcgaccacaccccgtccagcattctcagtcctcacacctgcatcccaggaccgtgggggccggtcgtcatttccctcttgaacatgtgctccgaagtataactctgggacctactgcccgtctctctcttccaccaggttcctgcatgaggagccctgatcaactggatcaccactttgcccagcctctgaacaccatgcaccaggcctcaatatcccagttccctttggccttttagttacaggtgaatgctgagaatgtgtcagagacaagtgcagcagcagcgatggttggtagtatagatcatttactcttcagacaattcccaaacctccattagtccaagagtttctacatcttcctccccagcaagaggcaacgtcaagtgatgaatttcccccctttactctgcctctgctccccatttgctagtttgaggaagtgacatagaggagaagccagctgtaggggcaagagggaaatgcaagtcacctgcaggaatccagctagatttggagaagggaatgaaactaacattgaatgactaccatggcacgctaaatagtatcttgggtgccaaattcatgtatccacttagctgcattggtccagggcatgtcagtctggatacagccttacctccaggtagcacttaactggtccattcacctagactgcaagtaagaagacaaaatgactgagaccgtgtgcccacctgaacttattgtctttacttggcctgagctaaaagcttgggtgcaggacctgtgtaactagaaagttgcctacttcagaacctccagggcgtgagtgcaaggtcaaacatgactggcttccaggccgaccatcaatgtaggaggagagctgatgtggagggtgacatgggggctgcccatgttaaacctgagtccagtgctctggcattgggcagtcacggttaaagccaagtcatgtgtgtctcagctgtttggaggtgatgattttgcatcttccaagcctcttcaggtgtgaatctgtggtcaggaaaacacaagtcctaatggaacccttaggggggaaggaaatgaagattccctataacctctgggggtggggagtaggaataaggggccttgggcctccataaatctgcaatctgcaccctcctcctagagacagggagatcgtgttctgctttttacatgaggagcagaactgggccatacacatgttcaagaactaggggagctacctggtagcaagtgagtgcagacccacctcaccttgggggaatctcaaactcataggcctcagatacacgatcacctgtcatatcaggtgagcactggcctgcttggggagagacctgggcccctccaggtgtaggaacagcaacactcctggctgacaactaagccaatatggccctaggtcattcttgcttccaatatgcttgccactccttaaatgtcctaatgatgagaaactctctttctgaccaattgctatgtttacataacacgcatgtactcatgcatcccttgccagagcccatatatgtatgcatatataaacatagcactttttactacatagctcagcacattgcaaggtttgcatttaagttaaaaaaaaaaaaaaaaaaaaactaaaggtgaaagatgccacattgaacaaactaaattcccaacccggttctggcaaagaatccagttatcccttccatgaagacgcacataactctcttacttggtctttccattagggacaacataagtcttgttttacatcaaataaaaacaatgttaaaaagtgtgtgaaccttaaaaatggaagtctactagtttacatacctacttcagaggacatggaaatgaccatgggcctgcatttcagggaccaaagcaaattaggcctggcctaaaatacatcagaccttttgtaagaaagaatttcaataaagcaaaaaacatgtcacaag

SEQ ID NO: 11 - DNA, 1968 bp, Homo sapiens
tagaggagctgccggtgcctcctcagaacatctcctgatcgctacccaggaccaggcaccaaggacagggagtcccaggcgcacaccccccattctgggtcccccaggcccagacccccactctgccacaggttgcatcttgacctggtcctcctgcagaagtggcccctgtggtcctgctctgagactcgtccctgggcgcccctgcagcccctttctatgactccatctggatttggctggctgtggggacgcggtccgaggggcggcctggctctcagcgtggtggcagccagctctctggccaccatggcaaatgctgagatctgaggggacaaggctctacagcctcagccaggggcactcagctgttgcagggtgtgatggagaacaaagctatgtacctacacaccgtcagcgactgtgacaccagctccatctgtgaggattcctttgatggcaggagcctgtccaagctgaacctgtgtgaggatggtccatgtcacaaacggcgggcaagcatctgctgtacccagctggggtccctgtcggccctgaagcatgctgtcctggggctctacctgctggtcttcctgattcttgtgggcatcttcatcttagcagtgtccaggccgcgcagctcccctgacgacctgaaggccctgactcgcaatgtgaaccggctgaatgagagcttccgggacttgcagctgcggctgctgcaggctccgctgcaagcggacctgacggagcaggtgtggaaggtgcaggacgcgctgcagaaccagtcagactcgttgctggcgctggcgggcgcagtgcagcggctggagggcgcgctatgggggctgcaggcgcaggcggtgcagaccgagcaggcggtggccctgctgcgggaccgcacgggccagcagagcgacacggcgcagctggagctctaccagctgcaggtggagagcaacagtagccagctgctgctgaggcgccacgcgggcctgctggacgggctggcgcgcagggtgggcatcctgggcgaggagctggccgacgtgggcggcgtgctgcgcggcctcaaccacagcctgtcctacgacgtggccctccaccgcacgcggctgcaggacctgcgggtgctggtgagcaacgccagcgaggacacgcgccgcctgcgcctggcgcacgtaggcatggagctgcagctgaagcaggagctggccatgctcaacgcggtcaccgaggacctgcgcctcaaggactgggagcactccatcgcactgcggaacatctccctcgcgaaagggccaccgggacccaaaggtgatcagggggatgaaggaaaggaaggcaggcctggcatccctggattgcctggacttcgaggtctgcccggggagagaggtaccccaggattgcccgggcccaagggcgatgatgggaagctgggggccacaggaccaatgggcatgcgtgggttcaaaggtgaccgaggcccaaaaggagagaaaggagagaaaggagacagagctggggatgccagtaaggacattctgctggggccgtgggatatggtgttggcacagggctagctgtcccccgagcagccccataagtttggaggttcagaggctggagccatggctggggctcaagtgtcaaaggaggctccctaccttttttagggctctgctggtctagcaagagatgctgatagaccccaggggcactggccacatttctagaggtgtcataaacctggcggttgtgtgcatggatctggaggcttcccccggtcactcgctagcccagctggtataatctctgtgcctcagtgttctcatctataaaatagggataacaggagtctttaccttataaggtcattgtgaaaattgaatgagttaatctgtgtaaagtgcttatgatcatgctggacacctggtgaggaaaaaaaaaaaaaaaaaaaaaaaa

SEQ ID NO: 12 - DNA, 848 bp, Homo sapiens
tttatacggactggcggcgagagcagctgcagttcgcatctcaggcagtacctagaggagctgcagctgaagcaggagctggccatgctcaacgcggtcaccgaggacctgcgcctcaaggactgggagcactccatcgcactgcggaacatctccctcgcgaaagggccaccgggacccaaaggtgatcagggggatgaaggaaaggaaggcaggcctggcatccctggattgcctggacttcgaggtctgcccggggagagaggtaccccaggattgcccgggcccaagggcgatgatgggaagctgggggccacaggaccaatgggcatgcgtgggttcaaaggtgaccgaggcccaaaaggagagaaaggagagaaaggagacagagctggggatgccagtggcgtggaggccccgatgatgatccgcctggtgaatggctcaggtccgcacgagggccgcgtggaagtgtaccacgaccggcgttggggcaccgtgtgtgacgacggctgggacaagaaggacggagacgtggtgtgccgcatgctcggcttccgcggtgtggaggaggtgtaccgcacagctcgattcgggcaaggcactgggaggatctggatggatgacgttgcctgcaagggcacagaggaaaccatcttccgctgcagcttctccaaatggggggtgacaaactgtggacatgccgaagatgccagcgtgacatgcaacagacactgaaagtgggcagagcccaagttcggggtcctgcacagagcacccttcctgcatccctggggtggggcacagctcggggccaccctgaccatgcctcgaccacaccccgtcca

SEQ ID NO: 13 - DNA, 567 bp, Homo sapiens
actcgcaatgtgaaccggctgaatgagagcttccgggacttgcagctgcggctgctgcaggctccgctgcaagcggacctgacggagcaggtgtggaaggtgcaggacgcgctgcagaaccagtcagactcgttgctggcgctggcgggcgcagtgcagcggctggagggcgcgctgtggggcctgcgcctggcgcacgtaggcatggagctgcagctgaagcaggagctggccatgctcaacgcggtcaccgaggacctgcgcctcaaggactgggagcactccatcgcactgcggaacatctccctcgcgaaagggccaccgggacccaaaggtgatcagggggatgaaggaaaggaaggcaggcctggcatccctggattgcctggacttcgaggtctgcccggggagagaggtaccccaggattgcccgggcccaagggcgatgatgggaagctgggggccacaggaccaatgggcatgcgtgggttcaaaggtgaccgaggcccaaaaggagagaaaggagagaaaggagacagagctggggatgccagtggcgtggaggcc

SEQ ID NO: 14 - DNA, 1074 bp, Homo sapiens
atggagaacaaagctatgtacctacacaccgtcagcgactgtgacaccagctccatctgtgaggattcctttgatggcaggagcctgtccaagctgaacctgtgtgaggatgtgtccaggccgcgcagctcccctgacgacctgaaggccctgactcgcaatgtgaaccggctgaatgagagcttccgggacttgcagctgcggctgctgcaggctccgctgcaagcggacctgacggagcaggtgtggaaggtgcaggacgcgctgcagaaccagtcagactcgttgctggcgctggcgggcgcagtgcagcggctggagggcgcgctgtgggggctgcaggcgcaggcggtgcagaccgagcaggcggtggccctgctgcgggaccgcacgggccagcagagcgacacggcgcagctggagctctaccagctgcaggtggagagcaacagtagccagctgctgctgaggcgccacgcgggcctgctggacgggctggcgcgcagggtgggcatcctgggcgaggagctggccgacgtgggcggcgtgctgcgcggcctcaaccacagcctgtcctacgacgtggccctccaccgcacgcggctgcaggacctgcgggtgctggtgagcaacgccagcgaggacacgcgccgcctgcgcctggcgcacgtaggcatggagctgcagctgaagcaggagctggccatgctcaacgcggtcaccgaggacctgcgcctcaaggactgggagcactccatcgcactgcggaacatctccctcgcgaaagggccaccgggacccaaaggtgatcagggggatgaaggaaaggaaggcaggcctggcatccctggattgcctggacttcgaggtctgcccggggagagaggtaccccaggattgcccgggcccaagggcgatgatgggaagctgggggccacaggaccaatgggcatgcgtgggttcaaaggtgaccgaggcccaaaaggagagaaaggagagaaaggagacagagctggggatgccagtaaggacattctgctggggccgtgggatatggtgttggcacagggctag

(19) United States
(12) Patent Application Publication
(10) Pub. No.: US 2013/0137106 A1
(43) Pub. Date: May 30, 2013

(54) Tumor Marker and Methods of Use Thereof

(71) Applicants: Renata Grifantini, Siena (IT); Piero Pileri, Siena (IT); Susanna Campagnoli, Siena (IT); Matteo Parri, Siena (IT); Alberto Grandi, Siena (IT); Andrea Pierleoni, Siena (IT)

(72) Inventors: Renata Grifantini, Siena (IT); Piero Pileri, Siena (IT); Susanna Campagnoli, Siena (IT); Matteo Parri, Siena (IT); Alberto Grandi, Siena (IT); Andrea Pierleoni, Siena (IT)

(73) Assignee: EXTERNAUTICS S.P.A., Siena (IT)

(21) Appl. No.: 13/695,359

(22) PCT Filed: Apr. 29, 2011
(86) PCT No.: PCT/EP2011/056825

§ 371 Date: Feb. 7, 2013

(30) Foreign Application Priority Data
Apr. 30, 2010 (EP) 10161559.9

(51) Int. Cl. C07K 14/705; C07K 7/08; C07K 16/30; G01N 33/68; C12Q 1/68

(52) U.S. Cl. CPC C07K 14/705; C07K 16/30; C07K 7/08
USPC 435/6.12; 530/350; 536/23.5; 435/7.23; 530/387.9; 530/326

(57) ABSTRACT

Newly identified proteins as markers for the detection of breast, colon, lung and ovary tumors, or as therapeutic targets for their treatment, affinity ligands capable of selectively interacting with the newly identified markers and methods for tumor diagnosis and therapy using such ligands.

The present invention relates to a newly identified protein as marker for the detection of tumors, or as targets for their treatment, particularly of tumors affecting lung, colon, breast and ovary. Also provided are affinity ligands capable of selectively interacting with the newly identified markers, as well as methods for tumor diagnosis and therapy using such ligands.

### BACKGROUND OF THE INVENTION

Tumor Markers (or Biomarkers)

Tumor markers are substances that can be produced by tumor cells or by other cells of the body in response to cancer. In particular, a protein biomarker is either a single protein or a panel of different proteins that could be used to unambiguously distinguish a disease state. Ideally, a biomarker would have both a high specificity and sensitivity, being represented in a significant percentage of the cases of given disease and not in healthy state.

Biomarkers can be identified in different biological samples, like tissue biopsies or preferably biological fluids (saliva, urine, blood-derivatives and other body fluids), whose collection does not necessitate invasive treatments. Tumor marker levels may be categorized in three major classes on the basis of their clinical use. Diagnostic markers can be used in the detection and diagnosis of cancer. Prognostics markers are indicative of specific outcomes of the disease and can be used to define predictive models that allow the clinicians to predict the likely prognosis of the disease at time of diagnosis. Moreover, prognosis markers are helpful to monitor the patient response to a drug therapy and facilitate a more personalized patient management. A decrease or return to a normal level may indicate that the cancer is responding to therapy, whereas an increase may indicate that the cancer is not responding. After treatment has ended, tumor marker levels may be used to check for recurrence of the tumor. Finally, therapeutic markers can be used to develop tumor-specific drugs or affinity ligand (i.e. antibodies) for a tumor treatment.

Currently, although an abnormal tumor marker level may suggest cancer, this alone is usually not enough to accurately diagnose cancer and their measurement in body fluids is frequently combined with other tests, such as a biopsy and radioscopic examination. Frequently, tumor marker levels are not altered in all of people with a certain cancer disease, especially if the cancer is at early stage. Some tumor marker levels can also be altered in patients with noncancerous conditions. Most biomarkers commonly used in clinical practice do not reach a sufficiently high level of specificity and sensitivity to unambiguously distinguish a tumor from a normal state.

To date the number of markers that are expressed abnormally is limited to certain types/subtypes of cancer, some of which are also found in other diseases. (http://www.cancer.gov/cancertopics/factsheet).

For example, prostate-specific antigen (PSA) levels are often used to screen men for prostate cancer, but this is controversial since elevated PSA levels can be caused by both prostate cancer or benign conditions, and most men with elevated PSA levels turn out not to have prostate cancer.

Another tumor marker, Cancer Antigen 125, (CA 125), is sometimes used to screen women who have an increased risk for ovarian cancer. Scientists are studying whether measurement of CA 125, along with other tests and exams, is useful to find ovarian cancer before symptoms develop. So far, CA 125 measurement is not sensitive or specific enough to be used to screen all women for ovarian cancer. Mostly, CA 125 is used to monitor response to treatment and check for recurrence in women with ovarian cancer. Finally, human epidermal growth factor receptor (HER2) is a marker protein overproduced in about 20% of breast cancers, whose expression is typically associated with a more aggressive and recurrent tumors of this class.

Routine Screening Test for Tumor Diagnosis

Screening tests are a way of detecting cancer early, before there are any symptoms. For a screening test to be helpful, it should have high sensitivity and specificity. Sensitivity refers to the test's ability to identify people who have the disease. Specificity refers to the test's ability to identify people who do not have the disease. Different molecular biology approaches such as analysis of DNA sequencing, small nucleotide polymorphyms, in situ hybridization and whole transcriptional profile analysis have done remarkable progresses to discriminate a tumor state from a normal state and are accelerating the knowledge process in the tumor field. However so far different reasons are delaying their use in the common clinical practice, including the higher analysis complexity and their expensiveness. Other diagnosis tools whose application is increasing in clinics include in situ hybridization and gene sequencing.

Currently, Immuno-HistoChemistry (IHC), a technique that allows the detection of proteins expressed in tissues and cells using specific antibodies, is the most commonly used method for the clinical diagnosis of tumor samples. This technique enables the analysis of cell morphology and the classification of tissue samples on the basis of their immunoreactivity. However, at present, IHC can be used in clinical practice to detect cancerous cells of tumor types for which protein markers and specific antibodies are available. In this context, the identification of a large panel of markers for the most frequent cancer classes would have a great impact in the clinical diagnosis of the disease.

Anti-Cancer Therapies

In the last decades, an overwhelming number of studies remarkably contributed to the comprehension of the molecular mechanisms leading to cancer. However, this scientific progress in the molecular oncology field has not been paralleled by a comparable progress in cancer diagnosis and therapy. Surgery and/or radiotherapy are still the main modality of local treatment of cancer in the majority of patients. However, these treatments are effective only at initial phases of the disease and in particular for solid tumors of epithelial origin, as is the case of colon, lung, breast, ovary, prostate and others, while they are not effective for distant recurrence of the disease. In some tumor classes, chemotherapeutic treatments have been developed, which generally relies on drugs, hormones and antibodies, targeting specific biological processes used by cancers to grow and spread. However, so far many cancer therapies had limited efficacy due to severity of side effects and overall toxicity. Indeed, a major effort in cancer therapy is the development of treatments able to target specifically tumor cells causing limited damages to surrounding normal cells thereby decreasing adverse side effects. Recent developments in cancer therapy in this direction are encouraging, indicating that in some cases a cancer specific therapy is feasible. In particular, the development and commercialization of humanized monoclonal antibodies that recognize specifically tumor-associated markers and promote the elimination of cancer is one of the most promising solution that appears to be an extremely favorable market opportunity for pharmaceutical companies. However, at present the number of therapeutic antibodies available on the market or under clinical studies is very limited and restricted to specific cancer classes. So far licensed monoclonal antibodies currently used in clinics for the therapy of specific tumor classes show only a partial efficacy and are frequently associated with chemotherapies to increase their therapeutic effect. Administration of Trastuzumab (Herceptin), a commercial monoclonal antibody targeting HER2 in conjunction with Taxol adjuvant chemotherapy induces tumor remission in about 42% of the cases (1). Bevacizumab (Avastin) and Cetuximab (Erbitux) are two monoclonal antibodies recently licensed for use in humans, targeting the endothelial and epithelial growth factors respectively that, combined with adjuvant chemotherapy, proved to be effective against different tumor diseases. Bevacizumab proved to be effective in prolonging the life of patients with metastatic colorectal, breast and lung cancers. Cetuximab demonstrated efficacy in patients with tumor types refractory to standard chemotherapeutic treatments (1).

In summary, available screening tests for tumor diagnosis are uncomfortable or invasive and this sometimes limits their applications. Moreover tumor markers available today have a limited utility in clinics due to either their incapability to detect all tumor subtypes of the defined cancers types and/or to distinguish unambiguously tumor vs. normal tissues. Similarly, licensed monoclonal antibodies combined with standard chemotherapies are not effective against the majority of cases. Therefore, there is a great demand for new tools to advance the diagnosis and treatment of cancer.

Experimental Approaches Commonly Used to Identify Tumor Markers

Most popular approaches used to discover new tumor markers are based on genome-wide transcription profile or total protein content analyses of tumor. These studies usually lead to the identification of groups of mRNAs and proteins which are differentially expressed in tumors. Validation experiments then follow to eventually single out, among the hundreds of RNAs/proteins identified, the very few that have the potential to become useful markers. Although often successful, these approaches have several limitations and often, do not provide firm indications on the association of protein markers with tumor. A first limitation is that, since frequently mRNA levels not always correlate with corresponding protein abundance (approx. 50% correlation), studies based on transcription profile do not provide solid information regarding the expression of protein markers in tumor (2, 3, 4, 5).

A second limitation is that neither transcription profiles nor analysis of total protein content discriminate post-translation modifications, which often occur during oncogenesis. These modifications, including phosphorylations, acetylations, and glycosylations, or protein cleavages influence significantly protein stability, localization, interactions, and functions (6).

As a consequence, large scale studies generally result in long lists of differentially expressed genes that would require complex experimental paths in order to validate the potential markers. However, large scale genomic/proteomic studies reporting novel tumor markers frequently lack of confirmation data on the reported potential novel markers and thus do not provide solid demonstration on the association of the described protein markers with tumor.

Approach Used to Identify the Protein Marker Included in the Present Invention

The approach that we used to identify a tumor marker is based on an innovative immuno-proteomic technology. In essence, a library of recombinant human proteins has been produced from E. coli and it is used to generate polyclonal antibodies against each of the recombinant proteins.

The screening of the antibodies library on Tissue microarrays (TMAs) carrying clinical samples from different patients affected by the tumor under investigation leads to the identification of specific tumor marker proteins. Therefore, by screening TMAs with the antibody library, the tumor markers are visualized by IHC, the classical technology applied in all clinical pathology laboratories. Since TMAs also include healthy tissues, the specificity of the antibodies for the tumors can be immediately appreciated and information on the relative level of expression and cellular localization of the markers could be obtained. In our approach the markers are subjected to a validation process consisting in a molecular and cellular characterization.

Altogether, the selective detection of the marker protein in tumor samples and the subsequent validation experiments lead to an unambiguous confirmation of the marker identity and confirm its association with defined tumor classes. Moreover this experimental process provides an indication of the possible use of the protein as tools for diagnostic or therapeutic intervention. For instance, a protein showing a cell surface localization could be both diagnostic and therapeutic marker, against which both chemical and antibody therapies can be developed. Differently, a marker showing a cytoplasmic localization could be more likely considered for the development of tumor diagnostic tests and chemotherapy/small molecules treatments.

### SUMMARY OF THE INVENTION

The present invention provides new means for the detection and treatment of breast, colon, lung and ovary tumors, based on the identification of Scavenger receptor class A member 5 (SCARA 5) marker specific for these tumor types.

In preferred embodiments, the invention provides the use of SCARA5 as marker or target for breast, colon, lung, ovary tumors.

The invention also provides a method for the diagnosis of these cancer types, comprising a step of detecting the above-identified marker in a biological sample, e.g. in a tissue sample of a subject suspected of having or at risk of developing malignancies or susceptible to cancer recurrences. In addition, the tumor marker identifies a novel target for affinity ligands which can be used for therapeutic applications. Also provided are affinity ligands, particularly antibodies, capable of selectively interacting with the newly identified protein marker expressed on the cell surface. The antibodies can be used to specifically discriminate cancer cells, based on the recognition of SCARA5. Moreover, antibodies able to detect SCARA5 on the cell surface can be used to directly kill or promote killing of cancer cells either as unconjugated or conjugated with cell payloads (e.g. radioisotopes, drugs, or toxins).

State of the Art

Despite the involvement of SCARA5 in tumor has been partially investigated, so far no previous evidence clearly documents the association of SCARA5 with breast, lung, ovary and colon tumors.

Recently, SCARA5 has been studied in Hepatocellular Carcinoma (HCC) (8). In this tumor type SCARA5 has been reported to be a tumor suppressor gene, whose expression was frequently downregulated as a result of promoter hypermethylation and allelic imbalance. Furthermore, SCARA5 knockdown via RNA interference markedly enhanced HCC cell growth in vitro, colony formation in soft agar, and invasiveness, tumorigenicity, and lung metastasis in vivo. By contrast, SCARA5 overexpression suppressed these malignant behaviors. Moreover, SCARA5 was found to physically associate with focal adhesion kinase (FAK), a non-receptor tyrosine kinase, and modulate tyrosine phosphorylation. FAK is known to be important in the regulation of focal adhesion dynamics and disassembly during cell migration. It is activated in a range of tumor cells, and its increased activity correlates with the malignancy and invasiveness of human HCC and other tumors. The interaction of SCARA5 with FAK inhibits the tyrosine phosphorylation cascade of the FAK-Src-Cas signaling pathway. Conversely, silencing SCARA5 stimulated the signaling pathway via increased phosphorylation of certain tyrosine residues of FAK, Src, and p130Cas; it was also associated with activation of MMP9, a tumor metastasis-associated enzyme. Taken together, these experimental data indicate that SCARA5 overexpression inhibits tumorigenicity, cell invasion, and metastasis; on the contrary, SCARA5 knockdown enhances tumorigenicity, cell invasion, and tumor metastasis in vivo via activation of the FAK signaling pathway.

SCARA5 has been included in some patent applications on specific types of cancer.

A European patent application (EP2159291A1, publication date 29 Jan. 2010) reports SCARA5 as a non-priority target in a set of 30 signature genes, selected on the basis of transcription profile analysis in breast cancer tumors and claimed as diagnosis tool for these cancer types. Another international application (WO2008104543A2) includes SCARA5 within a list of approximately 300 differentially expressed genes in bone tissue metastasis of patients affected by breast cancer. There is no mention to a possible use of these genes for the diagnosis of breast tumor but they are claimed as predictive of occurrence of metastasis of breast cancer. In both patent applications, evidences are limited to RNA analysis and no experimental confirmation is provided on the expression of SCARA5 protein in these tumor types. Moreover, the data support the diagnostic/predictive value of a signature/cluster of genes, while no evidence is provided on the possibility to exploit the transcript level of SCARA5, as single gene, for the indicated purpose.

SCARA5 is included in a patent application (WO2009093213A2, international publication date 23 Sep. 2009) disclosing five clusters of genes useful for predicting or diagnosing outcome of brain tumor treatment. In this application, SCARA5 is reported as a non-priority member of one of five clusters of genes whose high transcription level indicates a better outcome to chemo-radiotherapy treatment of brain tumors. Also in this case, data are limited to RNA expression in brain tumor samples with respect to the normal tissues, while no evidence of protein differential expression is reported.

SCARA5 is also mentioned in a US patent application (US20090047689A1 filed 2008 Jun. 20) related to autoantigen biomarkers for early diagnosis of lung adenocarcinoma. The application is focused on the identification of a recognition profile of serum from patients affected by lung adenocarcinoma which is proposed as a tool for tumor diagnosis. A panel of 133 proteins (autoantigens) was identified, showing higher reactivity with serum IgGs from patients affected by lung adenocarcinoma, compared to healthy individuals. In this long list of proteins is included SCARA5. However, there is no evidence supporting the use of individual autoantigens for the diagnosis. Moreover, no data are given on the over-expression of SCARA5 in tumor samples. Finally, the applications is not related to the identification of SCARA5 in tumor samples.

SCARA5 is also mentioned in WO201002467, describing a method to determine the percentage of breast tumor cells based on the transcription pattern of a set of 35 genes by Q-RT-PCR or similar types of analysis. A signature of 13 genes (not including SCARA5), is indicated as preferred for the method. In this patent application, the use of the entire gene signature (major evidences are provided for the 13 genes) is required to assess the amount of breast cancer cells in a given sample. WO201002467 provides no evidence that any of the listed genes can be used as single gene for the indicated purpose, nor this can be inferred from the data therein reported. Furthermore, there is no evidence that may allow to predict (i) the diagnostic value of the transcription level of SCARA5, as a single gene, and (ii) the use of SCARA5 protein as biomarker to distinguish tumor from healthy state.

None of the cited publications provide sufficient evidences that expression of SCARA5 protein is associated with tumor, in that: i) in hepatocellular carcinoma, high SCARA5 expression is inversely correlated with a tumor state; ii) patent application data reporting the differential expression of SCARA5 in breast and lung cancers are only based on transcription profile analysis of tumor versus healthy states, but no supportive data are provided confirming the presence of SCARA5 protein in these tumors; as reported in the previous section, frequently there is no correlation between mRNA and protein expression level, iii) the patent application describing SCARA5 as autoantigen, able to induce antibodies in patients affected by lung cancer, does not provide evidences on the over-expression of SCARA5 in tissues of these tumor type. Differently, we describe SCARA5 as a tumor biomarker and report the possibility of using a diagnostic method based on the direct detection of this protein; iv) none of the cited patent applications (reporting either increase of SCARA5 transcript or elicitation of antibodies in patients) shows the diagnostic property of SCARA5 when used individually; v) finally, none of the reported studies provide indication on the possible use of SCARA5 as a target for therapeutic intervention.

### DISCLOSURE OF THE INVENTION

The present invention is based on the surprising finding that antibodies specific for SCARA5 are able to specifically stain breast, colon, lung, ovary tumor tissues from patients, while negative or very poor staining is observed in normal lung tissues from the same patients. These antibodies have been found to specifically bind to a protein for which no previous association with tumor has been reported.

According to the present invention SCARA5 is provided as a protein marker for breast, colon, lung and ovary tumors and in general for cancers of these types. As described below, an antibody generated towards the SCARA5 protein shows a selective immunoreactivity in histological preparation of breast, colon, lung and ovary cancer tissues which indicates the presence of SCARA5 in these cancer samples and makes SCARA5 protein and its antibody highly interesting tools for specifically distinguishing these cancer types from a normal state. Moreover, an antibody generated against SCARA5 is able to specifically recognize the protein on the surface of cancer cell lines, indicating that this proteins could be developed as a target for anti-cancer therapies.

Hence, in a first aspect, the invention provides a marker for breast, colon, lung and ovary tumors which is selected from:

(i) SCARA5 protein, in one of its isoforms SEQ ID NO:1, SEQ ID NO:2, SEQ ID NO:3, SEQ ID NO:4, SEQ ID NO:5, SEQ ID NO:6 or SEQ ID NO:7, or a different isoform having sequence identity of at least 80%, preferably at least 90%, more preferably at least 95% to SEQ ID NO:1, SEQ ID NO:2, SEQ ID NO:3, SEQ ID NO:4, SEQ ID NO:5, SEQ ID NO:6 or SEQ ID NO:7;

(ii) a nucleic acid molecule containing a sequence coding for a SCARA5 protein, said encoding sequence being preferably SEQ ID NO:8, SEQ ID NO:9, SEQ ID NO:10, SEQ ID NO:11, SEQ ID NO:12, SEQ ID NO:13 or SEQ ID NO:14;

As used herein the “% amino acid sequence identity” with respect to the marker protein sequences identified herein indicates the percentage of amino acid residues in a protein variant or isoform, or in a portion thereof, that are identical to the amino acid residues in the specific marker sequence, after aligning the sequences and introducing gaps, if necessary, to achieve the maximum percent sequence identity, and not considering any conservative substitution as part of the sequence identity.

Identity between nucleotide sequences is preferably determined by the Smith-Waterman homology search algorithm as implemented in the SSEARCH program (Oxford Molecular), using an affine gap search with parameters gap open penalty=12 and gap extension penalty=1.

Scavenger receptor class A member 5 (SCARA 5; synonyms: Tesr; NET33; FLJ23907; MGC45780; GeneID: ENSG00000168079.

SCARA5 transcripts and variants

| SCARA5 - isorfoms | Transcript ID | Length (bp) | Protein ID | Length (aa) |
|---|---|---|---|---|
| SCARA5 - isoform 1 | ENST00000354914 | 4026 | ENSP00000346990 | 495 |
| SCARA5 - isoform 2 | ENST00000524352 | 1945 | ENSP00000428663 | 400 |
| SCARA5 - isoform 3 | ENST00000301906 | 1514 | ENSP00000301906 | 357 |
| SCARA5 - isoform 4 | ENST00000380385 | 2934 | ENSP00000369746 | 270 |
| SCARA5 - isoform 5 | ENST00000541268 | 848 | ENSP00000440110 | 217 |
| SCARA5 - isoform 6 | ENST00000517320 | 567 | ENSP00000427902 | 189 |
| SCARA5 - isoform 7 | ENST00000518030 | 1074 | ENSP00000430713 | 357 |

is a plasma membrane protein acting as a ferritin receptor that mediates non-transferrin-dependent delivery of iron. It mediates cellular uptake of ferritin-bound iron by stimulating ferritin endocytosis from the cell surface with consequent iron delivery within the cell. Delivery of iron to cells by ferritin is required for the development of specific cell types, suggesting the existence of cell type-specific mechanisms of iron traffic in organogenesis, which alternatively utilize transferrin or non-transferrin iron delivery pathways (7).

A further aspect of this invention is a method of screening a tissue sample for malignancy, which comprises determining the presence in said sample of the above-mentioned tumor marker. This method includes detecting either the marker protein, e.g. by means of labeled monoclonal or polyclonal antibodies that specifically bind to the target protein, or the respective mRNA, e.g. by means of polymerase chain reaction techniques such as RT-PCR. The methods for detecting proteins in a tissue sample are known to one skilled in the art and include immunoradiometric, immunoenzymatic or immunohistochemical techniques, such as radioimmunoassays, immunofluorescent assays or enzyme-linked immunoassays. Other known protein analysis techniques, such as polyacrylamide gel electrophoresis (PAGE), Western blot or Dot blot are suitable as well. Preferably, the detection of the protein marker is carried out with the immune-histochemistry technology, particularly by means of High Through-Put methods that allow the analyses of the antibody immune-reactivity simultaneously on different tissue samples immobilized on a microscope slide. Briefly, each Tissue Micro Array (TMA) slide includes tissue samples suspected of malignancy taken from different patients, and an equal number of normal tissue samples from the same patients as controls. The direct comparison of samples by qualitative or quantitative measurement, e.g. by enzimatic or colorimetric reactions, allows the identification of tumors.

In one embodiment, the invention provides a method of screening a sample of lung, colon, breast or ovary tissue for malignancy, which comprises determining the presence in said sample of the SCARA5 protein tumor marker, variants or isoforms thereof as described above.

A further aspect of the invention is a method in vitro for determining the presence of a lung, colon, breast or ovary tumor in a subject, which comprises the steps of:
providing a sample of the tissue suspected of containing tumor cells;
determining the presence of a SCARA5 tumor marker in said tissue sample by detecting the expression of the marker protein or the presence of the respective mRNA transcript;

wherein the detection of the tumor marker in the tissue sample is indicative of the presence of tumor in said subject.

The methods and techniques for carrying out the assay are known to one skilled in the art and are preferably based on immunoreactions for detecting proteins and on PCR methods for the detection of mRNAs. The same methods for detecting proteins or mRNAs from a tissue sample as disclosed above can be applied.

A further aspect of this invention is the use of the SCARA5 tumor marker herein provided as target for the identification of candidate antitumor agents. Accordingly, the invention provides a method for screening compounds which comprises contacting cells expressing SCARA5 protein with the test compound and determining the binding of said compound to said tumor-associated protein. In addition, the ability of the test compound to modulate the activity of each target molecule can be assayed.

A further aspect of the invention is an antibody or a fragment thereof, which is able to specifically recognize and bind to one of the tumor-associated proteins described above. The term “antibody” as used herein refers to any type of immunoglobulins, including IgG, IgM, IgA, IgD and IgE. Such antibodies may include polyclonal, monoclonal, chimeric, single chain, antibodies or fragments such as Fab or scFv. The antibodies may be of various origin, including human, mouse, rat, rabbit and horse, or chimeric antibodies. The production of antibodies is well known in the art. For the production of antibodies in experimental animals, various hosts including goats, rabbits, rats, mice, and others, may be immunized by injection with polypeptides of the present invention or any fragment or oligopeptide or derivative thereof which has immunogenic properties or forms a suitable epitope. Monoclonal antibodies may be produced following the procedures described in Kohler and Milstein, Nature 265:495 (1975) or other techniques known in the art.

The antibodies to the tumor markers of the invention can be used to detect the presence of the marker in histologic preparations or to distinguish tumor cells from normal cells. To that purpose, the antibodies may be labeled with radiocative, fluorescent or enzyme labels.

In addition, the antibodies can be used for treating proliferative diseases by modulating, e.g. inhibiting or abolishing the activity of a target protein according to the invention. Therefore, in a further aspect the invention provides the use of antibodies to SCARA5 protein for the preparation of a therapeutic agent for the treatment of proliferative diseases. For use in therapy, the antibodies can be formulated with suitable carriers and excipients, optionally with the addition of adjuvants to enhance their effects.

A further aspect of the invention relates to a diagnostic kit containing suitable means for detection, in particular SCARA5 polypeptides or polynucleotides, antibodies or fragments or derivatives thereof described above, reagents, buffers, solutions and materials needed for setting up and carrying out the immunoassays, nucleic acid hybridization or PCR assays described above. Parts of the kit of the invention can be packaged individually in vials or bottles or in combination in containers or multicontainer units.

### DESCRIPTION OF THE FIGURES

FIG. 1. Analysis of purified SCARA5 recombinant protein expressed in E. coli

Left panel: Comassie staining of purified His-tag SCARA5 fusion protein expressed in E. coli separated by SDS-PAGE. Arrow marks the protein band of the expected size. Molecular weight markers are reported on the left.

FIG. 2. Staining of breast tumor TMAs with anti-SCARA5 antibodies

Examples of TMA of breast tumor (lower panel) and normal tissue samples (upper panel) stained with anti-SCARA5. The antibody stains specifically tumor cells (in dark gray).

FIG. 3. Staining of colon tumor TMAs with anti-SCARA5 antibodies

Examples of TMA of colon tumor (lower panel) and normal tissue samples (upper panel) stained with anti-SCARA5 antibodies. The antibody stains specifically tumor cells (in dark gray).

FIG. 4. Staining of lung tumor TMAs with anti-SCARA5 antibodies

Examples of TMA of lung tumor (lower panel) and normal tissue samples (upper panel) stained with anti-SCARA5 antibodies. The antibody stains specifically tumor cells (in dark gray).

FIG. 5. Staining of ovary tumor TMAs with anti-SCARA5 antibodies

Examples of TMA of ovary tumor (lower panel) and normal tissue samples (upper panel) stained with anti-SCARA5 antibodies. The antibody stains specifically tumor cells (in dark gray).

FIG. 6. SCARA5 expression and localization in transiently transfected HeLa cells. A) Western blot analysis of SCARA5 expression in total protein extracts from HeLa cells (corresponding to 2×10⁵ cells) transfected with the empty vector pcDNA3 (lane 1) or with the plasmid construct encoding the SCARA5 gene (lane 2) stained with a specific antibody. Two bands of approximately 60 and 70 KDa were visible in HeLa cells transfected with plasmid expressing the SCARA5 495 amino acid-protein isoform, while the same bands were not visible in HeLa cells transfected with the empty pcDNA3 plasmid. The 60 KDa protein species shows the expected molecular mass, while the 70 KDa species likely corresponds to a SCARA5 form carrying post-translational modifications (e.g. glycolyslation). Arrow marks the expected SCARA5 protein band. Molecular weight markers are reported on the left. B) Flow cytometry analysis of SCARA5 surface localization in HeLa cells transfected with the plasmid construct encoding the SCARA5 gene (panel 2), compared to control cells transfected with the empty vector pcDNA3 (panel 1). X axis, Fluorescence scale; Y axis, Side Scatter scale C) Confocal microscopy analysis of SCARA5 surface localization in HeLa cells transfected with the with the plasmid construct encoding the SCARA5 gene (2) or with empty vector pcDNA3, as control (1) and stained with anti-SCARA5 antibodies and DAPI to visualize the nuclei. Left and right panels represent the surface and intracellular cell staining, respectively. The SCARA5 specific staining accumulated at the surface of transfected cells while no staining was detected in control HeLa cells (marked by arrows).

FIG. 7. Expression and localization of SCARA5 in tumor cell lines

A) Western blot analysis of SCARA5 expression. Total protein extracts (corresponding to 2×10⁵ cells) from breast (MDA-MB231 and SKBr3), colon (HCT15 and Colo205), ovary (OVCAR3, OVCAR4, OVCAR5, OVCAR8) and lung (H226) tumor cell lines were separated by SDS-PAGE, transferred onto nitrocellulose membranes and probed with anti-SCARA5 antibodies. Molecular weight markers are reported on the left. B) Flow cytometry analysis of SCARA5 surface localization in the OVCAR8 cell line stained with the anti-SCARA5 (white peak) or unrelated antibodies (grey peak). X axis, Fluorescence scale; Y axis, Cells (expressed as % relatively to major peaks). C) Confocal microscopy analysis of SCARA5 surface localization OVCAR8 tumor cell line stained with anti-SCARA5 (right panel) or irrelevant antibodies (left panel) and DAPI to visualize the nuclei. Arrow marks the SCARA5 staining visible at the cell surface.

FIG. 8. Detection of SCARA5 in lung tumor tissue homogenates by immunoblot. Immunoblot analysis of tissue homogenates from biopsies of lung tumor (lanes 4, 5, 6) and corresponding normal samples (lanes 1, 2, 3) stained with an anti-SCARA antibody. Molecular weight markers are reported on the left. Different protein species ranging from 40 to 20 KDa were specifically detected by the antibody, compatible with annotated SCARA5 isoforms.

FIG. 9. Anti-SCARA5 rabbit polyclonal antibodies detect bands of expected size in Flp-In-293 showing stable expression of the three encoded SCARA5 variants.

Western blot analysis of total protein extracts (corresponding to 2×10⁵ cells) from Flp-In 293 stable clones expressing SCARA5 isoform 1 (lane 2), isoform 2 (lane 3) and isoform 4 (lane 4) and Flp-In 293 stably transfected with control pcDNA5 vector. Molecular weight markers are reported on the left.

FIG. 10. The monoclonal antibody mAb61 specifically recognize SCARA5 isoform 2

Western blot analysis of total protein extracts (corresponding to 2×10⁵ cells) from Flp-In 293 stable clones expressing SCARA5 isoform 1 (lane 2), isoform 2 (lane 3) and isoform 4 (lane 4) and Flp-In 293 stably transfected with control pcDNA5 vector. mAb61 specifically stains SCARA5 protein bands in cells expressing isoform 2 of the protein while isoforms 1 and 4 are nor detected. Molecular weight markers are reported on the left.

FIG. 11. The monoclonal antibody mAb61 specifically stains the surface of the Flp-In 293 cells expressing the SCARA5 isoform 2

A. Flow cytometry analysis in control 293 Flip-in cells (left graph) and SCARA5-is2 293 Flip-in cells (right graph) stained with a negative control antibody (filled curve) or with the anti-SCARA5 monoclonal antibody (empty curve). X axis, Fluorescence scale; Y axis, Cells (expressed as % relatively to major peaks); This indicate the cell surface localization of SCARA5 and that the monoclonal antibody specifically recognizes an epitope of SCARA5 exposed on the cell surface.

B. Confocal microscopy analysis. The anti-SCARA5 monoclonal antibody stains the plasma membrane of 293 Flip-in cells expressing SCARA5 iso2 (right panels), with (lower panels) or without (upper panels) cell permeabilization with the detergent. No binding was observed on cells transfected with the empty pcDNA5 vector (left panels) or the SCARA5 iso1 (middle panels).

FIG. 12. Identification of the protein sequence recognized by the anti-SCARA5 monoclonal antibody. Sequence alignment of SCARA5 protein isoforms. As shown in the figure a unique peptide sequence distinguishes the protein isoforms 2, 3, and 7 from the other isoforms, corresponding to the region recognized by the monoclonal antibody mAb61.

### EXAMPLES

### Example 1

### Generation of Recombinant SCARA5 and Anti-SCARA5 Antibodies to Detect the Expression of SCARA5 in Tumor Samples

Methods

The entire coding region or suitable fragments of the SCARA5 gene encoding the target protein, were designed for cloning and expression using bioinformatic tools with the human genome sequence as template (Lindskog M et al (2005). The leader sequence for secretion was replaced with the ATG codon to drive the expression of the recombinant proteins in the cytoplasm of E. coli. For cloning, genes were PCR-amplified from cDNAs mixtures generated from pools of total RNA derived from Human testis, Human placenta, Human bone marrow, Human fetal brain, using specific primers. Clonings were designed so as to fuse a 10 histidine tag sequence at the 3′ end, annealed to in house developed vectors, derivatives of vector pSP73 (Promega) adapted for the T4 ligation independent cloning method (9) and used to transform E. coli NovaBlue cells recipient strain. E. coli tranformants were plated onto selective LB plates containing 100 μg/ml ampicillin (LB Amp) and positive E. coli clones were identified by restriction enzyme analysis of purified plasmid followed by DNA sequence analysis. For expression, plasmids were used to transform BL21-(DE3) E. coli cells and BL21-(DE3) E. coli cells harbouring the plasmid were inoculated in ZYP-5052 growth medium (10) and grown at 37° C. for 24 hours. Afterwards, bacteria were collected by centrifugation, lysed into B-Per Reagent containing 1 mM MgCl2, 100 units DNAse I (Sigma), and 1 mg/ml lysozime (Sigma). After 30 min at room temperature under gentle shaking, the lysate was clarified by centrifugation at 30.000 g for 40 min at 4° C. Proteins were purified from the inclusion bodies by resuspending the pellet coming from lysate centrifugation in 40 mM TRIS-HCl, 1 mM TCEP {Tris(2-carboxyethyl)-phosphine hydrochloride, Pierce} and 6M guanidine hydrochloride, pH 8 and performing an IMAC in denaturing conditions. Briefly, the resuspended material was clarified by centrifugation at 30.000 g for 30 min and the supernatant was loaded on 0.5 ml columns of Ni-activated Chelating Sepharose Fast Flow (Pharmacia). The column was washed with 50 mM TRIS-HCl buffer, 1 mM TCEP, 6M urea, 60 mM imidazole, 0.5M NaCl, pH 8. Recombinant proteins were eluted with the same buffer containing 500 mM imidazole. Proteins were analysed by SDS-Page and their concentration was determined by Bradford assay using the BIORAD reagent (BIORAD) with a bovine serum albumin standard according to the manufacturer's recommendations.

To generate antisera, the purified SCARA5 recombinant proteins were used to immunize CD1 mice (6 week-old females, Charles River laboratories, 5 mice per group) intraperitoneally, with 3 protein doses of 20 micrograms each, at 2 week-interval. Freund's complete adjuvant was used for the first immunization, while Freund's incomplete adjuvant was used for the two booster doses. Two weeks after the last immunization animals were bled and sera collected from each animal was pooled.

Results

Gene fragments of the expected size were obtained by PCR from cDNA generated from pools of total RNA derived from Human testis, Human placenta, Human bone marrow, Human fetal brain, using primers specific for SCARA5 gene.

A fragment of the transcript ENST00000301906 encoding a protein of 318 residues, corresponding to the amino acid region from 40 to 357 of ENSP00000301906 sequence was obtained.

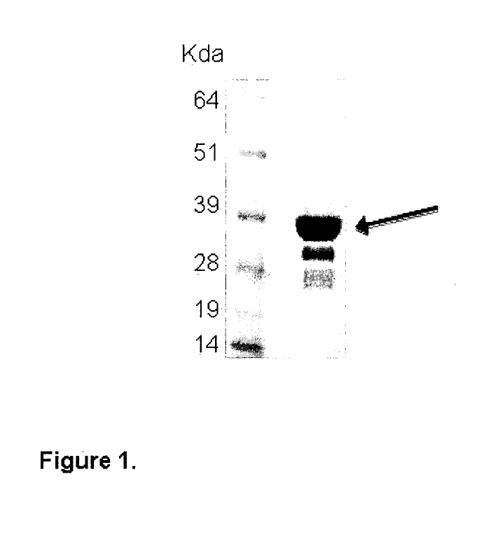

A clone encoding the correct amino acid sequence was identified and, upon expression in E. coli, a protein of the correct size was produced and subsequently purified using affinity chromatography (FIG. 1).

### Example 2

### Tissue Profiling by Immune-Histochemistry

Methods

The analysis of the antibody capability to recognize their target proteins in tumor samples was carried out by Tissue Micro Array (TMA), a miniaturized immuno-histochemistry technology suitable for HTP analysis that allows to analyse the antibody immuno-reactivity simultaneously on different tissue samples immobilized on a microscope slide.

Since the TMAs include both tumor and healthy tissues, the specificity of the antibodies for the tumors can be immediately appreciated. The use of this technology, differently from approaches based on transcription profile, has the important advantage of giving a first hand evaluation on the potential of the markers in clinics. Conversely, since mRNA levels not always correlate with protein levels (approx. 50% correlation), studies based on transcription profile do not provide solid information regarding the expression of protein markers.

A tissue microarray was prepared containing formalin-fixed paraffin-embedded cores of human tissues from patients affected by breast, colon, lung and ovary cancers and corresponding normal tissues as controls and subsequently analyzed using the specific antibody sample. For each tumor class the TMA design consisted in 10 pathological and 10 normal tissue samples from 5 well pedigreed patients (equal to two tumor samples and 2 normal tissues from each patient) to identify promising target molecules differentially expressed in cancer and normal cells. The direct comparison between tumor and normal tissues of each patient allowed the identification of antibodies that stain specifically tumor cells and provided indication of target expression in lung tumor.

All formalin fixed, paraffin embedded tissues used as donor blocks for TMA production were selected from the archives at the IEO (Istituto Europeo Oncologico, Milan). Corresponding whole tissue sections were examined to confirm diagnosis and tumor classification, and to select representative areas in donor blocks. Normal tissues were defined as microscopically normal (non-neoplastic) and were generally selected from specimens collected from the vicinity of surgically removed tumors. The TMA production was performed essentially as previously described (11, 12). Briefly, a hole was made in the recipient TMA block. A cylindrical core tissue sample (1 mm in diameter) from the donor block was acquired and deposited in the recipient TMA block. This was repeated in an automated tissue arrayer “Galileo TMA CK 3500” della dita BioRep (Milan) until a complete TMA design was produced. TMA recipient blocks were baked at 42 <0>C for 2 h prior to sectioning. The TMA blocks were sectioned with 2-3 mm thickness using a waterfall microtome (Leica), and placed onto poli-L-lysinated glass slides for immunohistochemical analysis. For automated immunohistochemistry, glass slides were incubated for 30′ min in 60° C., de-paraffinized in xylene (2×15 min) using the Bio-Clear solution (Midway. Scientific, Melbourne, Australia), and re-hydrated in graded alcohols. For antigen retrieval, slides were immersed 0.01 M Na-citrate buffer, pH 6.0 at 99° C. for 30 min Slides were placed in the Autostainer® (DakoCytomation) and endogenous peroxidase was initially blocked with 3% H2O2, for 5 min. Slides were then blocked in Dako Cytomation Wash Buffer containing 5% Bovine serum albumin (BSA) and subsequently incubated with mouse antibodies for 30′ (dilution 1:200 in Dako Real™ dilution buffer). After washing with DakoCytomation wash buffer, slides were incubated with the goat anti-mouse peroxidase conjugated Envision® for 30 min each at room temperature (DakoCytomation). Finally, diaminobenzidine (DakoCytomation) was used as chromogen and Harris hematoxylin (Sigma-Aldrich) was used for counterstaining. The slides were mounted with Pertex® (Histolab).

The staining results have been evaluated by a trained pathologist at the light microscope, and scored according to both the percentage of immunostained cells and the intensity of staining. The individual values and the combined score (from 0 to 300) were recorded in a custom-tailored database. Digital images of the immunocytochemical findings have been taken at a Leica DM LB light microscope, equipped with a Leica DFC289 color camera.

Results

A TMA design was obtained, including tumor tissue samples and normal tissues, derived from 5 patients affected by breast, colon, lung and ovary tumor. The results from tissue profiling showed that the antibodies specific for the recombinant proteins (see Example 1) are strongly immunoreactive on tissues from each of the four cancers, while no or poor reactivity was detected in normal tissues, indicating the presence of the target proteins in lung tumors. In most samples, the antibody staining accumulated at the plasma membrane of tumor cells. Based on this finding, the detection of SCARA5 protein in tumor tissue samples can be associated with lung breast, colon, lung and ovary tumors. Moreover, the SCARA5 localization at the plasma membrane makes this protein as a suitable target for therapies.

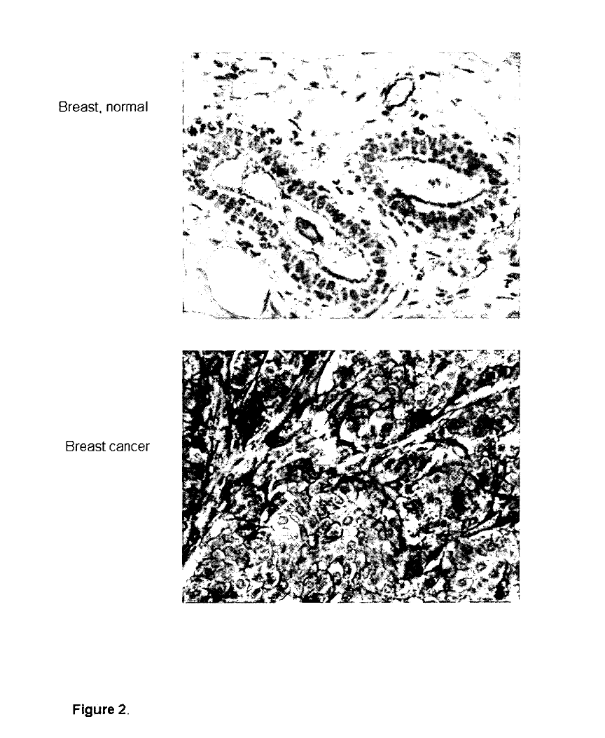

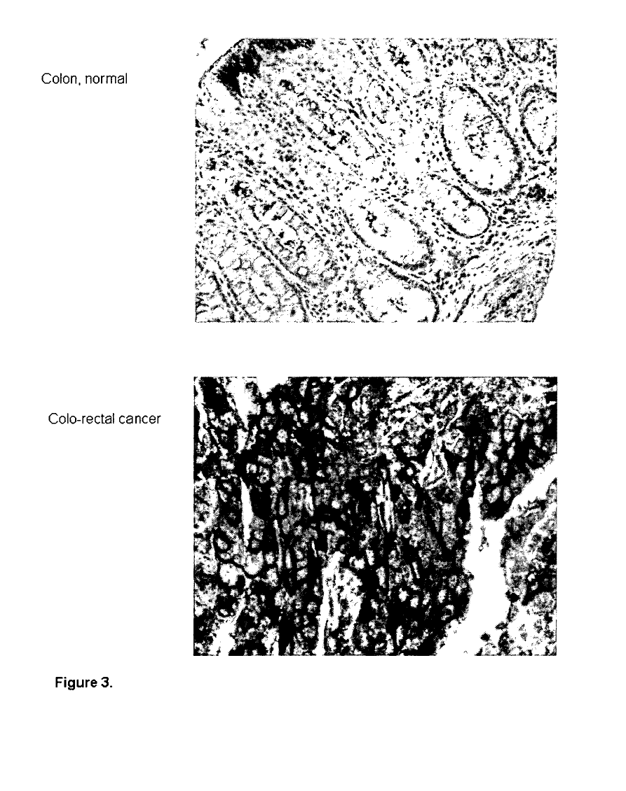

The capability of target-specific antibodies to stain the tumor tissues is summarized in Table 1. Representative examples of microscopic enlargements of tissue samples stained by the anti-SCARA5 antibody are reported in FIGS. 2-5.

The table reports the number of patients, out of the five screened, whose tumor tissue samples showed positive staining with the SCARA5-specific antibodies.

TABLE 1
Number of patients whose tumor tissues showed
positive immuno-histochemistry staining with the
anti-SCARA5 antibodies

| Tumor | Number of positive patients |
|---|---|
| Breast | 4/5 |
| Colon | 3/5 |
| Lung | 4/5 |
| Ovary | 2/5 |

### Example 3

### Confirmation of the Marker Association with the Tumor/s by Expanded IHC Analysis

Methods

The association of each protein with the indicated tumors was further confirmed on a larger collection of clinical samples for each tumor. To this aim, a tissue microarray was prepared for each of the four tumor classes containing 100 formalin-fixed paraffin-embedded cores of human tissues from 50 patients (equal to two tissue samples from each patient). The TMAs were stained with the SCARA5-antibodies, using the previously reported procedure. The staining results were evaluated, as above described, by a trained pathologist at the light microscope.

Results

Four TMA designs were obtained, for each of the four tumors, representing tissue samples from 50 patients. The results from tissue analysis showed that the anti-SCARA5 antibodies are strongly immune-reactive on a significant percentage of tissues from breast, colon, lung and ovary tumors indicating that the SCARA5 protein is are selectively detected in these tumor types. This finding confirms a strong association of SCARA5 marker with the indicated tumors. Table 2 reports the frequency of patients whose tumor tissue samples that showed positive IHC staining on the TMA.

TABLE 2
Frequency of patients whose tumor tissues showed positive
immuno-histochemistry staining with the anti-SCARA5 antibodies on the
expanded TMA analysis

| Tumor | Percentage of positive patients |
|---|---|
| Breast | 38 |
| Colon | 56 |
| Lung | 42 |
| Ovary | 34 |

### Example 4

### Expression and Localization of SCARA5 Protein in Transfected Mammalian Cells

Methods

SCARA5 expression was assessed by Western blot analysis on total protein extracts from eukaryotic cells transiently transfected with plasmid constructs containing the complete coding sequences of the genes encoding SCARA5 protein. Examples of this type of experiments are given for HeLa cells transfected with plasmid pcDNA3.1 in which one the annotated SCARA5 isoform (corresponding to Transcript ID ENST00000301906 and encoding a protein of 495 aminoacids) was cloned.

To this aim, cDNA were generated from pools of total RNA derived from Human testis, Human placenta, Human bone marrow, Human fetal brain, in reverse transcription reactions and the entire SCARA 5 coding region was PCR-amplified with specific primers pairs. PCR products were cloned into plasmid pcDNA3 (Invitrogen). HeLa cells were grown in DMEM-10% FCS supplemented with 1 mM Glutamine were transiently transfected with preparation of the resulting plasmid and with the empty vector as negative control using the Lipofectamine-2000 transfection reagent (Invitrogen). After 48 hours, cells were collected, lysed with PBS buffer containing 1% Triton X100 and expression of target proteins was assessed by Western blot analysis on total cell extracts (corresponding to 2×10⁵ cells) using anti-SCARA5 antibodies. Western blot was performed by separation of the protein extracts on pre-cast SDS-PAGE gradient gels (NuPage 4-12% Bis-Tris gel, Invitrogen) under reducing conditions, followed by electro-transfer to nitrocellulose membranes (Invitrogen) according to the manufacturer's recommendations. The membranes were blocked in blocking buffer composed of 1× PBS-0.1% Tween 20 (PBST) added with 10% dry milk, for 1 h at room temperature, incubated with the antibody diluted 1:2500 in blocking buffer containing 1% dry milk and washed in PBST-1%. The secondary HRP-conjugated antibody (goat anti-mouse immunoglobulin/HRP, Perkin Elmer) was diluted 1:5000 in blocking buffer and chemiluminescence detection was carried out using a Chemidoc-IT UVP CCD camera (UVP) and the Western lightning™ cheminulescence Reagent Plus (Perkin Elmer), according to the manufacturer's protocol.

The SCARA5 surface localization was assessed by Flow cytometry (FACS) and confocal microscopy analyses of HeLa transfected cells.

For Flow Cytometry analysis, HeLa cells transfected with each construct or with the empty vector (2×10⁴ per well) were pelletted in 96 U-bottom microplates by centrifugation at 200×g for 5 min at 4° C. and incubated for 1 hour at 4° C. with the appropriate dilutions of anti-SCARA5-antibodies. The cells were washed twice in PBS-5% FCS and incubated for 20 min with the appropriate dilution of R-Phycoerythrin (PE)-conjugated secondary antibodies (Jackson Immuno Research, Pa., USA) at 4° C. After washing, cells were analysed by a FACS Canto II flow cytometer (Becton Dickinson). Data were analyzed with FlowJo 8.3.3 program.

For confocal microscopy, transfected and control HeLa cells were plated on glass cover slips and after 48 h were washed with PBS and fixed with 3% formaldheyde solution in PBS for 20 min at RT. Then, after extensive washing in PBS, the cells were incubated with the antibodies overnight at 4° C. (1:200) with or without a previous permeabilization step with 0.01% BriJ96® (Fluka). Cells were then stained with Alexafluor 488-labeled goat anti-mouse antibodies (Molecular Probes). DAPI (Molecular Probes) was used to visualize nuclei. The cells were mounted with glycerol plastine and observed under a laser-scanning confocal microscope (LeicaSP5).

Results

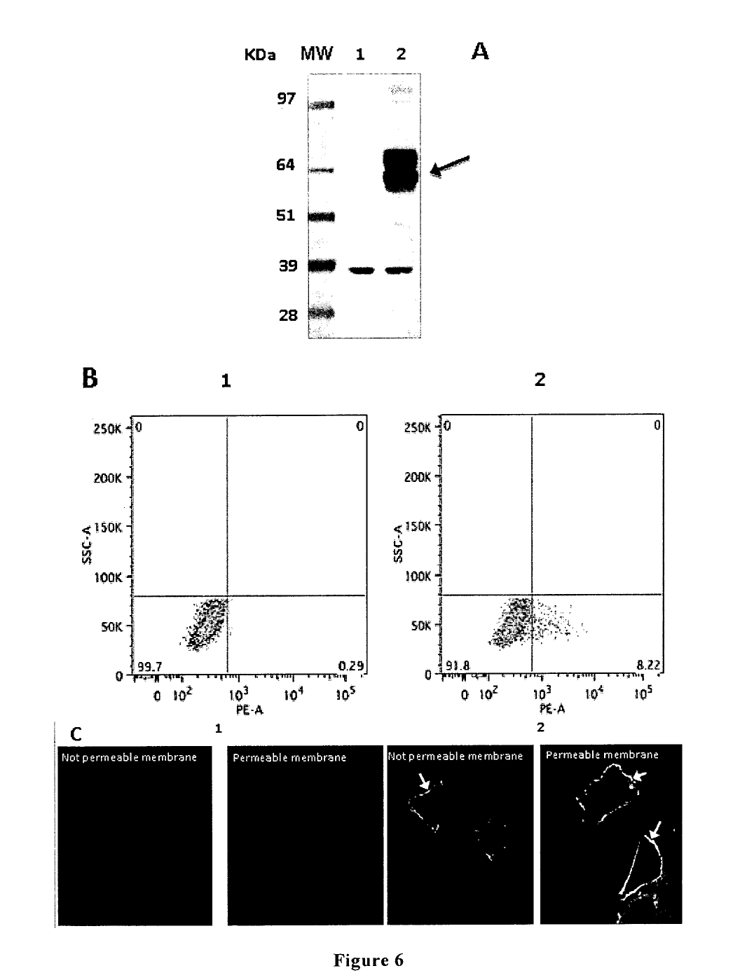

The complete coding sequence for the target protein SCARA5 was cloned in the eukaryotic expression vector pcDNA3.1, sequence-verified and used for transient transfection of HeLa cells. Expression of the target protein was detected by Western blot in total protein extracts from HeLa cells transfected with the SCARA5-encoding construct using the anti-SCARA5 antibody. Two bands of approximately 60 and 70 kDa were visible in HeLa cells transfected with plasmid expressing the SCARA5 495 amino acid-protein isoform while the same bands was not visible in HeLa cells transfected with the empty pcDNA3 plasmid. The 60 kDa protein species shows the expected molecular mass, while the 70 kDa species likely corresponds to a SCARA5 form carrying post-translational modifications (e.g. glycolyslation). This confirmed that the antibody recognized specifically its target protein. A second band of approximately 39 kDa was visible both in transfected and control HeLa cells, which could correspond to another SCARA5 isoform (known to exist), endogenously expressed by the cells. Results are represented in FIG. 6A.

Surface localization of target proteins was addressed by FACS and confocal microscopy analyses of transiently transfected cells stained with the specific antibodies. Data are reported for cells transfected with the construct encoding the SCARA5 495 amino acid isoform. In this experiment the SCARA5-antibody was capable of binding the surface of transfected HeLa cells, while no binding was observed on cells transfected with the empty pcDNA3 vector (FIG. 6B). A similar result was obtained when transfected HeLa cells were analysed by confocal microscopy. As shown in FIG. 6C, the anti-SCARA5 antibody was able to stain the plasma membrane of transfected cells expressing SCARA5, with or without cell permeabilization with the detergent. This indicates that this target protein is localized on the cell surface and is accessible to the external environment.

### Example 5

### Expression and Localization of SCARA5 Protein in Tumor Cell Lines

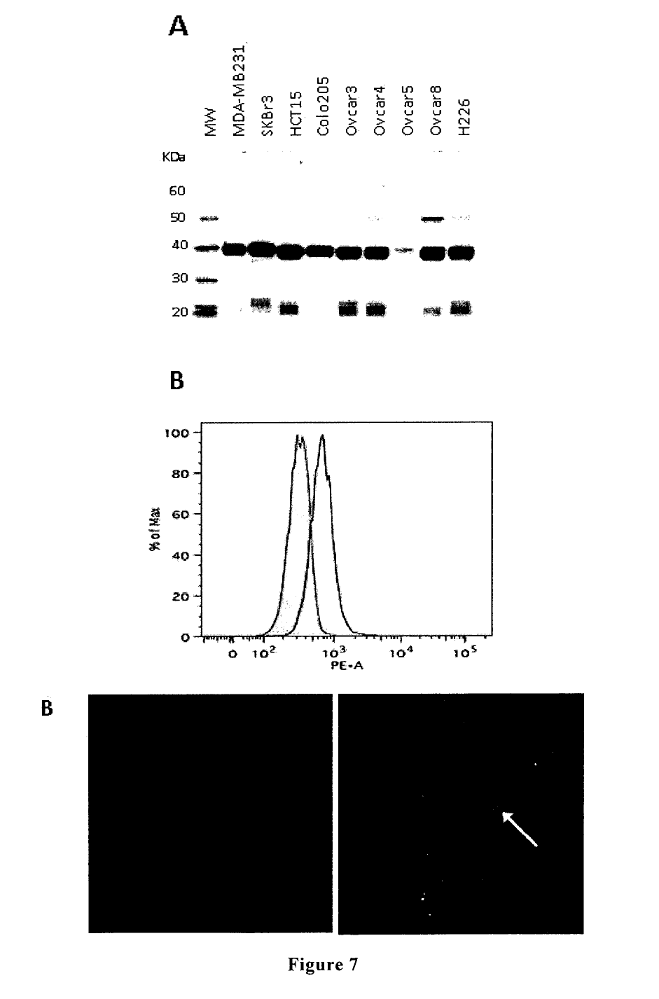

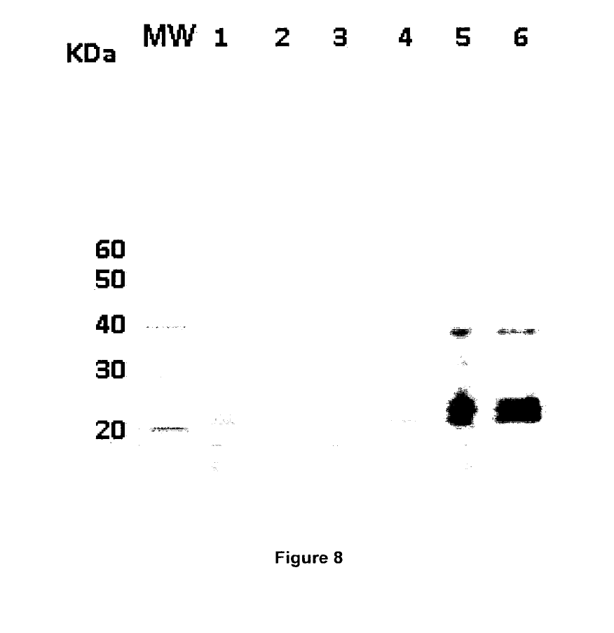

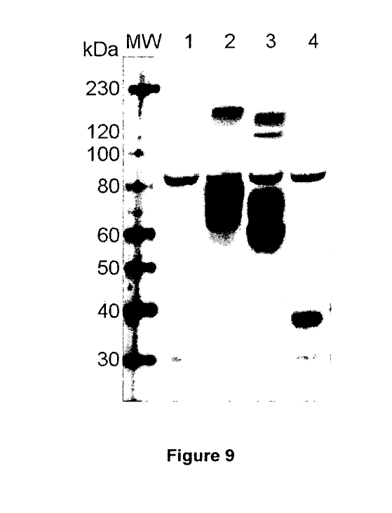

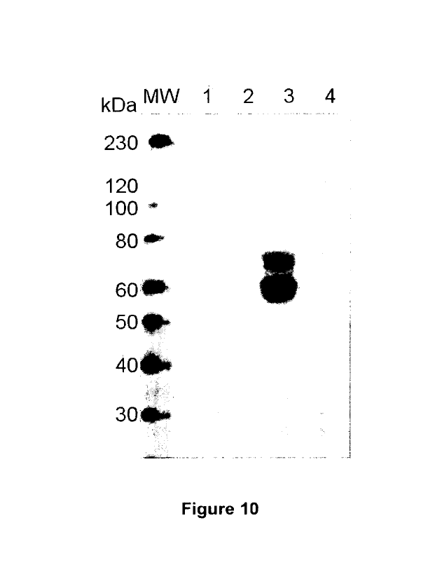

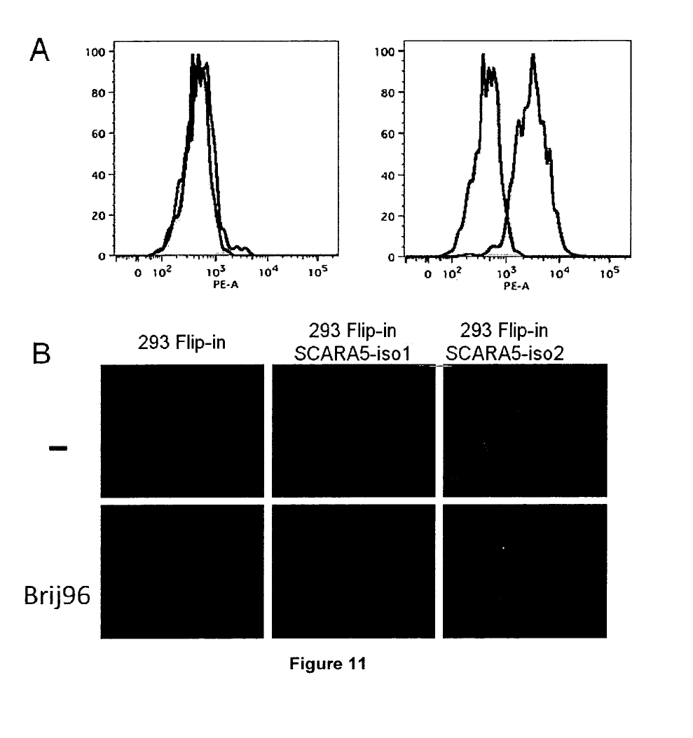

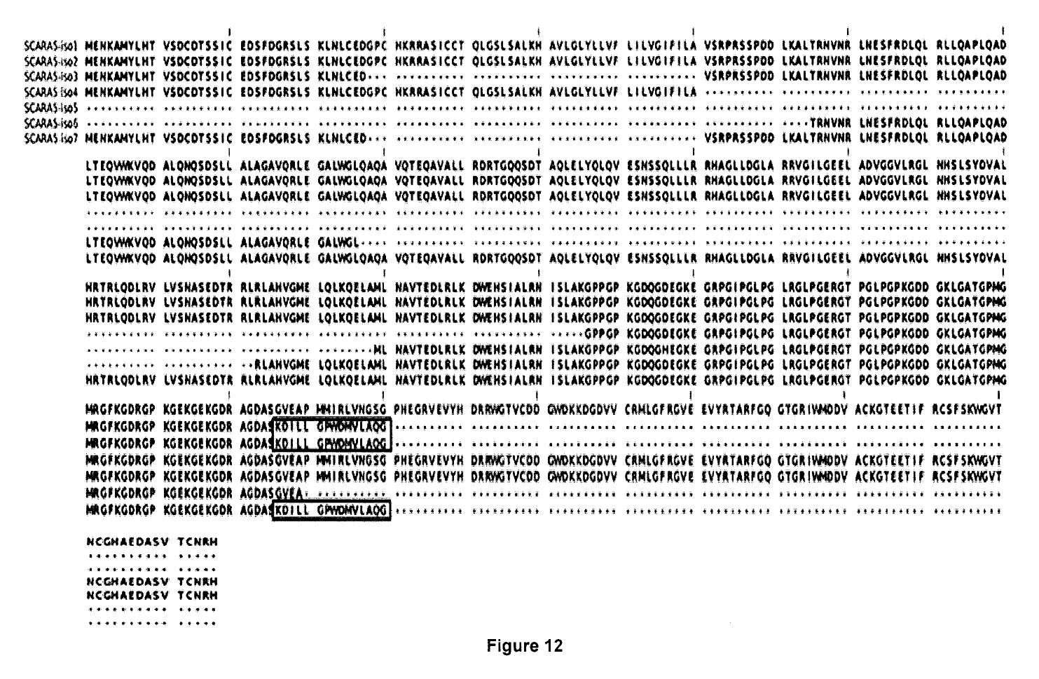

SCARA5 expression was also assessed by WB on total extracts from a panel of human epithelial cell lines representing the tumors analised by IHC. In each analysis, cells were cultured in under ATCC recommended conditions, and sub-confluent cell mono-layers were detached with PBS-0.5 mM EDTA and lysed by several freeze-thaw passages in PBS-1% Triton. Total protein extracts were loaded on SDS-PAGE (2×10⁵ cells/lane), and subjected to WB with specific antibodies as described above. (FIG. 7A)

The marker cellular localization was assessed by Flow cytometry and confocal microscopy analyses on tumor cell lines, using the above described procedures (see Example 5).

Results

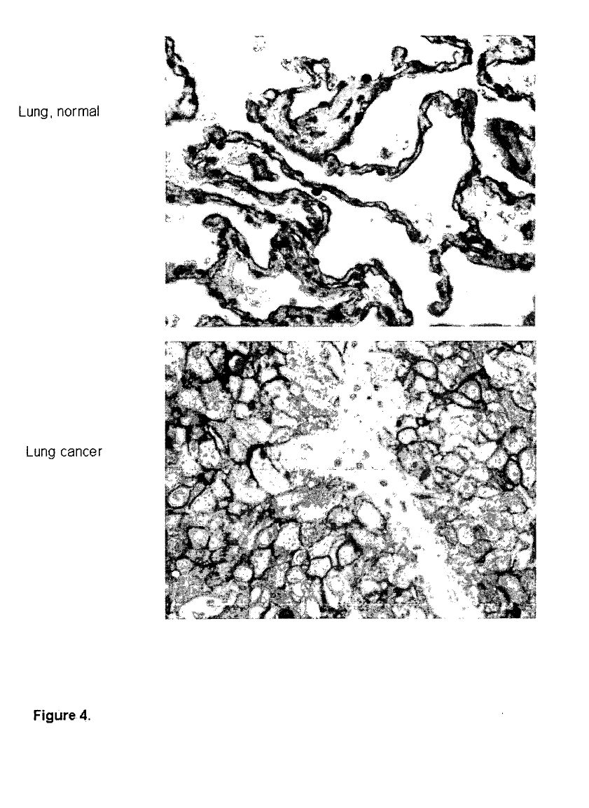

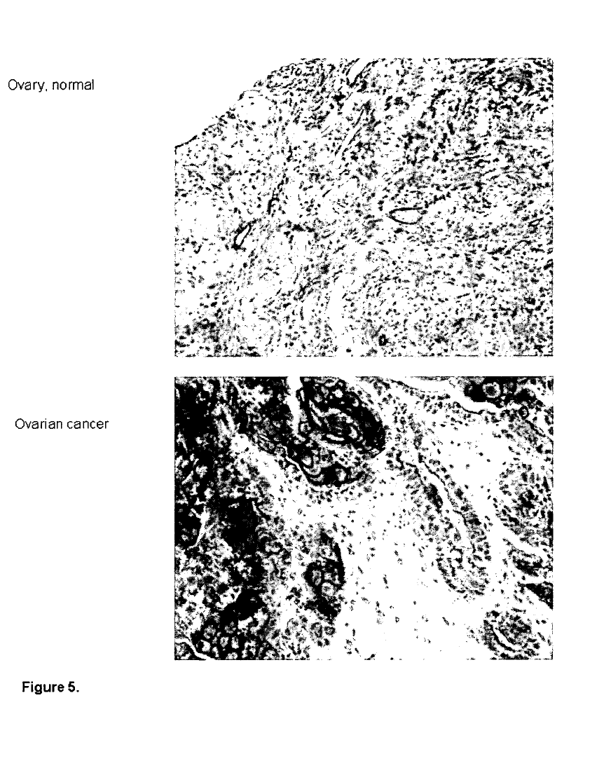

SCARA5 expression was confirmed in a panel of human tumor cell lines from breast, colon, lung and ovary tumors, examples of which are given in FIG. 4.

In particular, SCARA5 expression is reported for a panel of the tumor cell lines including OVCAR3, OVCAR4, OVCAR5, OVCAR8 (ovary adenocarcinoma), and HCT-15 and Colo205 (colorectal cancer), H226 (lung tumor), MDA-MB231 and SKBr3 (breast cancer). In all tested cell lines different protein species were detected by the antibody, including a major protein band of approximately 40 kDa and other proteins species of approximately 20, 50 and 60 kDa, that could correspond to the annotated SCARA5 isoforms (FIG. 7A). Surface staining of a panel of tumor cell lines indicates that SCARA5 protein is at least partially exposed on the surface of tested cells, as judged by the capability of the anti-SCARA5 antibody to bind the cell surface. An example of FACS analysis is given for the ovary tumor cell line OVCAR8 (FIG. 7B). FACS results were also confirmed by confocal microscopy analysis of the same tumor cell lines, among which OVCAR8 is represented in FIG. 7C. As shown in the figure, the anti-SCARA5 antibody was able to detect the protein on the cell surface (FIG. 7C). Both confocal microscopy and FACS data show that the protein is accessible to the external environment and this evidence suggests that SCARA5 could be exploited as therapeutic target of anticancer therapies.

### Example 6

### Detection of Target Protein in Tumor Tissue Homogenates

The presence of protein bands corresponding to SCARA5 protein was also investigated in tissue homogenates of tumor biopsies as compared to normal tissues from patients. Homogenates were prepared by mechanic tissue disruption in buffer containing 40 mM TRIS-HCl, 1 mM TCEP {Tris(2-carboxyethyl)-phosphine hydrochloride, Pierce) and 6M guanidine hydrochloride, pH 8. Western blot was performed by separation of the total protein extracts (20 μg/lane) proteins were detected by specific antibodies. An example of this analysis is given for the detection of SCARA5 in lung tumor homogenates

Results

The anti-SCARA5 antibodies detected specific protein bands in homogenates from tumor samples (ranging from 40 to 20 kDa), that could correspond to the annotated SCARA5 isoforms confirming the presence of the marker proteins in lung tumor. Results are reported in FIG. 8.

### Example 7

### Generation of Cell Lines Stably Over-Expressing SCARA5 Protein Variants on the Cell Surface

Stable cell lines over-expressing three SCARA5 isoforms predicted as surface exposed [namely isoform 1-ENSP00000346990 (iso1), isoform 2-ENSP00000428663 (iso2), and isoform 4-ENSP00000369746 (iso4)] were generated following the Flp-In system (Flp-In. System For Generating Stable Mammalian Expression Cell Lines by Flp Recombinase-Mediated Integration, Invitrogen) and used for expression and localization analysis. The Flp-In-293 Human embryonic kidney cell line (Invitrogen), previously shown to be negative for endogenous SCARA5 expression by immunoblot and flow cytometry, was used as recipient cell line. Briefly, the SCARA5 sequences encoding for selected isoforms were PCR-amplified from cDNAs mixtures generated from pools of total RNA (see Example 1) and cloned into vector pcDNA5-FRT. The resulting plasmids pcDNA5-SCARA5_iso1, pcDNA5-SCARA5_iso2 and pcDNA5-SCARA5_iso4 were used to transfect the Flp-In-293 cell line and clones showing stable SCARA5 expression were selected and maintained using Invitrogen recommended procedures. As negative control, stable clones were generated by transfecting the Flp-In-293 cell line using the empty pcDNA5 vector and selected clones (as described before) were used for the analysis. The correct expression of SCARA5 isoforms was assessed using anti-SCARA5 polyclonal antibodies by immunoblot analysis of total extracts from SCARA5-stable clones, using the above-described procedures. The expression and localization of SCARA5 isoforms were also analysed by confocal microscopy analysis.

Results

Anti-SCARA5 rabbit polyclonal antibodies were able to detect bands of expected size in Flp-In-293 showing stable expression of the three encoded SCARA5 variants, while no specific bands were detected in the mock control. Results are represented FIG. 9.

Confocal microscopy analysis of SCARA5 clones confirmed that the protein variants showed surface exposure and were accessible to antibody binding, similarly to what already found in transiently transfected HeLa cells (not shown). This confirmed that the SCARA5 stable clones reproduce the native SCARA5 localization and, therefore, can be used for investigations on SCARA5 and affinity ligands

### Example 8

### Generation of an Anti-SCARA5 Monoclonal Antibody Able to Detect the Protein on Flp-in-293 Stable Clone Over-Expressing Specific SCARA5 Isoforms

Monoclonal antibodies were generated from splenocytes of Balb/c mice immunized with SCARA5 recombinant protein through conventional myeloma-splenocyte somatic fusions and hybridoma screenings (13). Briefly, four- to six-week-old female BALB/c mice, were inoculated with the SCARA5 recombinant protein as described above (Example 1). Three days after the last immunization, animals were sacrificed and their spleen cells were fused with myeloma cells P3X63-Ag8.653 at a ratio of 5 spleen cells to 1 myeloma cell. After 2 week of incubation in HAT-selective medium, hybridoma supernatants were screened for Ab binding activity by surface staining of the Flp-In-293 clone stably expressing SCARA5 iso2, using by flow cytometry. Hybridomas secreting reactive antibodies were cloned by limiting dilution, and then expanded and frozen for subsequent use. Different cell lines were prepared from three fusions and characterized.

A cell line showing highly positive FACS staining was expanded and the cell culture supernatant was used for antibody purification by conventional protein G-Sepharose chromatography. The ability of the purified monoclonal antibody to recognize SCARA5 on the stable clone was further confirmed by FACS and confocal microscopy, as described in the examples above. Moreover, the ability of the monoclonal antibody to recognize the SCARA5 isoforms was verified by immunoblot.

Results

An anti-SCARA5 monoclonal antibody (namely mAb61) was generated and purified as described above. Immunoblot analysis of total extract from Flp-In 293 stable clones expressing SCARA5 isoforms 1, 2 and 4 showed that the antibody specifically detect SCARA5 and specifically recognize isoform 2 (FIG. 10). The antibody is able to specifically bind SCARA5 on the surface of the Flp-In 293 expressing the SCARA5 isoform 2 as defined by flow cytometry and confocal microscopy (FIG. 11).

In the flow cytometry assay the SCARA5 monoclonal antibody was capable of binding the surface 293 Flip-in cells expressing isoform 2 (FIG. 11A, right panel), while no binding was observed on cells transfected with the empty pcDNA5 vector (FIG. 11A, left panel) A similar result was obtained when transfected HeLa cells were analysed by confocal microscopy. As shown in FIG. 11B, the anti-SCARA5 monoclonal antibody was able to stain the plasma membrane of cells expressing the isoform 2 of SCARA5 (FIG. 11B, right panels), with (lower panels) or without (upper panels) cell permeabilization with the detergent. No binding was observed on cells transfected with the empty pcDNA5 vector (FIG. 11B, left panels) or the isoform 1 of SCARA5 (FIG. 11B, middle panels). This indicates that this monoclonal antibody specifically recognise the isoform 2 of SCARA5 protein localized on the cell surface and the epitope recognised is accessible from the outside.

Sequence alignment of SCARA5 isoforms allowed to identify the protein region recognized by the monoclonal antibody, that is included in the sequence KDILLGPWDMVLAQG (FIG. 12). This amino acid sequence is present in SCARA5 isoforms 2, 3 and 7 and not in isoforms 1, 4, 5, and 6. This indicates that the monoclonal antibody mAb61 can be used to detect three SCARA5 isoforms (2, 3 and 7) and discriminate them from other known variants in which the sequence is not present. Overall the data indicate that the anti-SCARA5 monoclonal antibody is particularly suitable to target tumor cells expressing SCARA5 on the cell surface. The antibody can be conveniently used for diagnostic applications based on the detection of tumor-associated SCARA5. In addition, it can be used for therapeutic applications, either unconjugated or conjugated with cell-payloads (e.g. toxins, drugs, etc.) able to promote killing of tumor cells.

### REFERENCES

1) Adams G. P. and Weiner L. M. (2005) Monoclonal antibody therapy cancer. Nat Biotechnol. 23:1147-1157.
2) Anderson, L., and Seilhamer, J. (1997). A comparison of selected mRNA and protein abundances in human liver. Electrophoresis 18: 533-537.
3) Chen, G., Gharib, T. G., Wang, H., Huang, C. C., Kuick, R., Thomas, D. G., Shedden, K. A., Misek, D. E., Taylor, J. M., Giordano, T. J., Kardia, S. L., Iannettoni, M. D., Yee, J., Hogg, P. J., Orringer, M. B., Hanash, S. M., and Beer, D. G. (2003) Protein profiles associated with survival in lung adenocarcinoma. Proc. Natl. Acad. Sci. U.S. A 100: 13537-13542.
4) Ginestier, C., Charafe-Jauffret, E., Bertucci, F., Eisinger, F., Geneix, J., Bechlian, D., Conte, N., Adelaide, J., Toiron, Y., Nguyen, C., Viens, P., Mozziconacci, M. J., Houlgatte, R., Birnbaum, D., and Jacquemier, J. (2002) Distinct and complementary information provided by use of tissue and DNA microarrays in the study of breast tumor markers. Am. J. Pathol. 161: 1223-1233.
5) Gygi, S. P., Rochon, Y., Franza, B. R., and Aebersold, R. (1999) Correlation between protein and mRNA abundance in yeast. Mol. Cell. Biol. 19, 1720-1730; Nishizuka, S., Charboneau, L., Young, L., Major, S., Reinhold, W. C., Waltham, M., Kouros-Mehr, H., Bussey, K. J., Lee, J. K., Espina, V., Munson, P. J., Petricoin, E., III, Liotta, L. A., and Weinstein, J. N. (2003) Proteomic profiling of the NCI-60 cancer cell lines using new high-density reverse-phase lysate microarrays. Proc. Natl. Acad. Sci. U.S.A. 100, 14229-14234.
6) Tyers, M., and Mann, M. (2003) From genomics to proteomics. Nature 422: 193-197.
7) Li J Y, Paragas N, Ned R M, Qiu A, Viltard M, Leete T, Drexler I R, Chen X, Sanna-Cherchi S, Mohammed F, Williams D, Lin C S, Schmidt-Ott K M, Andrews N C, Barasch J. SCARA5 is a ferritin receptor mediating non-transferrin iron delivery. Dev Cell. (2009) 16:35-46.
8) Huang J, Zheng D L, Qin F S, Cheng N, Chen H, Wan B B, Wang Y P, Xiao H S, Han Z G. Genetic and epigenetic silencing of SCARA5 may contribute to human hepatocellular carcinoma by activating FAK signaling. J Clin Invest. (2010) 120:223-41.
9) Aslanidis C, de Jong P J. (1990) Ligation-independent cloning of PCR products (LIC-PCR). Nucleic Acids Res. 18:6069-74.
10) Studier F W. (2005) Protein production by auto-induction in high density shaking cultures. Protein Expr Purif. 41:207-34.
11) Kononen J, Bubendorf L, Kallioniemi A, Bärlund M, Schraml P, Leighton S, Torhorst J, Mihatsch M J, Sauter G, Kallioniemi O P. Tissue microarrays for high-throughput molecular profiling of tumor specimens. (1998) Nat Med. 4:844-7.
12) Kallioniemi O P, Wagner U, Kononen J, Sauter G. (2001) Tissue microarray technology for high-throughput molecular profiling of cancer. Hum Mol Genet. 10:657-62.
13) Yang Jian, Shen Ming Hong Polyethylene Glycol-Mediated Cell Fusion, Springer Protocols, 2005, 325:59-66, DOI-10.1385/1-59745-005-7:59).

1. A tumor marker for use in the detection of breast, colon, lung and ovary cancer, wherein said tumor marker is:
(i) SCARA5 protein in one of its variant isoforms SEQ ID NO:1, SEQ ID NO:2, SEQ ID NO:3, SEQ ID NO:4, SEQ ID NO:5, SEQ ID NO:60R SEQ ID NO:7 or a different isoform having sequence identity of at least 80%, preferably at least 90%, more preferably at least 95% to SEQ ID NO:1, SEQ ID NO:2, SEQ ID NO:3, SEQ ID NO:4, SEQ ID NO:5, SEQ ID NO:6 or SEQ ID NO:7; or
(ii) a nucleic acid molecule containing a sequence coding for a SCARA5 protein as defined in (i), said encoding sequence being preferably SEQ ID NO:8, SEQ ID NO:9, SEQ ID NO:10, SEQ ID NO: 11, SEQ ID NO:12, SEQ ID NO:13, SEQ ID NO:14.

2. A method of screening a sample of breast, colon, lung or ovary tissue for malignancy, said method comprising determining the presence in said sample of a tumor marker according to claim 1.

3. A method according to claim 2, wherein said tumor marker is a SCARA5 protein, said method being based on immunoradiometric, immunoenzymatic or immunohistochemical techniques.

4. A method according to claim 2, wherein said tumor marker is a SCARA5 nucleic acid molecule, said method being based on polymerase chain reaction techniques.

5. A method in vitro for determining the presence of a breast, colon, lung or ovary tumor in a subject, comprising the steps of:
(a) providing a sample of the tissue suspected of containing tumor cells;
(b) determining the presence of a tumor marker according to claim 1 in said tissue sample by detecting the expression of the marker protein or the presence of the respective mRNA transcript;
wherein the detection of one or more tumor markers in the tissue sample is indicative of the presence of tumor in said subject.

6. A method of screening compounds for antitumor candidates, which comprises contacting cells expressing a tumor marker protein according to claim 1 with the test compound and determining the binding of said compound to said cells.

7. An antibody or a fragment thereof which is able to specifically recognize and bind the tumor marker protein according to claim 1.

8. An antibody according to claim 7, which is either monoclonal or polyclonal.

9. (canceled)

10. A diagnostic kit containing an antibody according to claim 7 and/or an oligonucleotide complementary to a nucleic acid molecule encoding a tumor marker according to claim 1, and optionally reagents, buffers, solutions and materials to carry out an immunoassay or a PCR assay.

11. An isolated peptide of SCARA5 protein, wherein said peptide is immunogenic and comprises the amino acid sequence KDILLGPWDMVLAQG (SEQ ID No: 15).

12. (canceled)

13. A monoclonal or polyclonal antibody which is able to specifically bind the peptide of claim 11.

14. (canceled)

15. An immunogenic composition containing the peptide of claim 11.
